SEQUENCE LISTING (13 sequences)

SEQ ID NO: 1 - DNA, 462 bp, Homo sapiens
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 2 - protein, 153 aa, Homo sapiens [sequence omitted]

SEQ ID NO: 3 - DNA, 423 bp, Mus musculus
atgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtag

SEQ ID NO: 4 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 5 - DNA, 414 bp, Equus caballus
atgggtctcacctaccaactgcttccagctctggtctgcttactagcatgtaccagcaacttcatccagggatgcaaatacgacatcaccttacaagagatcatcaaaacgctgaacaacctcacagatggaaagggcaagaattcgtgcatggagctgactgtagcggatgcctttgctggcccgaagaacacagatggaaaggaaatctgcagggctgcaaaggtgcttcaacagctctataaaagacatgacaggtccttgatcaaagaatgcctgagcggactggacaggaacctcaagggcatggcaaacgggacctgctgtactgtgaatgaagccaagaagagcacattgaaagactttttggaaaggctaaagacgatcatgagagagaaatactccaagtgttga

SEQ ID NO: 6 - protein, 137 aa, Equus caballus
MGLTYQLLPALVCLLACTSNFIQGCKYDITLQEIIKTLNNLTDGKGKNSCMELTVADAFAGPKNTDGKEICRAAKVLQQLYKRHDRSLIKECLSGLDRNLKGMANGTCCTVNEAKKSTLKDFLERLKTIMREKYSKC

SEQ ID NO: 7 - protein, 5 aa, Artificial Sequence
  Synthetic peptide sequence representing the enterokinase cleavage site
DDDDK

SEQ ID NO: 8 - DNA, 29 bp, Artificial Sequence
  Synthetic oligonucleotide primer
gcggccgcatgggtctcacctaccaactg

SEQ ID NO: 9 - DNA, 35 bp, Artificial Sequence
  Synthetic oligonucleotide primer
cgtcgtacagatcacacttggagtatttctctttc

SEQ ID NO: 10 - DNA, 31 bp, Artificial Sequence
  Synthetic oligonucleotide primer
ccggatccttatcgtcatcgtcgtacagatc

SEQ ID NO: 11 - protein, 937 aa, Homo sapiens
MGLTYQLLPALVCLLACTSNFIQGCKYDITLQEIIKTLNNLTDGKGKNSCMELTVADAFAGPKNTDGKEICRAAKVLQQLYKRHDRSLIKECLSGLDRNLKGMANGTCCTVNEAKKSTLKDFLERLKTIMREKYSKCDLYDDDDKDPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSFGPLASALQILDVSANPLHCACGAAFMDFLLEVQAAVPGLPSRVKCGSPGQLQGLSIFAQDLRLCLDEALSWDCFAL

SEQ ID NO: 12 - protein, 215 aa, Equus caballus
GTRDVVAAYNLTWKSTNFKTILEWEPKPINHVYTVEISPRLGNWKSKCFYTTDTECDLTDEVVKDVTGTYMARVSSYLANATSSAGDPPFTNAPEFTPFLETNLSQPTIQSFEQVGTKLNVTVQDARTLVRVNGTYLSLRDVFGKNLSYMLYYWKASSTGKKTAKTNTNEFLIDVDKGEDYCFNVQAVIPSRKTNQKSPESPIECTSQHKAVSRE

SEQ ID NO: 13 - protein, 147 aa, Bos taurus
DRDLDIDMSPKPTMFLPSITEIKHDNTGTYLCLLENFFPHVIKVYWREKRGNRVLPSQQGNTVKTADTYMKFSWLTVSENSMDKEHICIVKHEKNKRGDNQEILFPPVNEVVSSVVTATKPPNDGLKDKKKTSPCCNSTKACLKDEN

(19) United States
(12) Patent Application Publication
(10) Pub. No.: US 2011/0287454 A1
(43) Pub. Date: Nov. 24, 2011

(54) METHODS FOR MONOCLONAL ANTIBODY PRODUCTION

(75) Inventor: Bettina Wagner, Ithaca, NY (US)

(73) Assignee: CORNELL UNIVERSITY, Ithaca, NY (US)

(21) Appl. No.: 13/115,282

(22) Filed: May 25, 2011

Related U.S. Application Data
(63) Continuation-in-part of application No. PCT/US09/65669, filed on Nov. 24, 2009.
(60) Provisional application No. 61/117,832, filed on Nov. 25, 2008.

(51) Int. Cl. C12P 21/08; C07K 19/00; G01N 33/53; C12N 15/63

(52) U.S. Cl. 435/7.92; 435/70.21; 435/320.1; 530/351; 435/69.6

(57) ABSTRACT

This invention provides improved methods for production of monoclonal antibodies against a protein of interest. The present methods are based on immunization of an animal with a fusion protein between a protein of interest and a Th2 cytokine such as IL-4, IL-5, IL-13 and IL-31.

### CROSS-REFERENCE TO RELATED APPLICATIONS

This application is a continuation-in-part of International Application PCT/US09/65669, filed on Nov. 24, 2009, and claims the benefit of priority from U.S. Provisional Application No. 61/117,832, filed on Nov. 25, 2008.

### STATEMENT REGARDING FEDERALLY SPONSORED RESEARCH OR DEVELOPMENT

This invention was made with Government Support from U.S. Department of Agriculture under Contract No. 2006-35204-16880. The Government has certain rights in this invention.

### FIELD OF THE INVENTION

This invention relates to methods for efficient production of antibodies. In particular, the invention relates to improved methods for production of monoclonal antibodies against a protein of interest based on immunization with a fusion protein between a Th2 cytokine and the protein of interest.

### BACKGROUND OF THE INVENTION

Interleukin-4, or “IL-4”, is a pleiotropic cytokine produced by activated T cells. This cytokine is a ligand for interleukin 4 receptor (“IL-4R”). Among its many biological roles, IL-4 induces differentiation of naïve helper T cells (Th0 cells) to Th2 cells. Upon activation by IL-4, Th2 cells subsequently produce additional IL-4. IL-4 is also known to act as a B-cell stimulatory factor which induces antibody production. Naïve B-cells express IL-4R on their surface. When triggered by a specific antigen and IL-4, the naïve B-cells mature, proliferate and perform immunoglobulin class switching to IgG1.

While multiple recombinant expression systems are available, many proteins and peptides remain difficult to be produced and/or being secreted effectively in recombinant form and/or have low immunogenicity. These obstacles make it difficult to generate monoclonal antibodies against such proteins and peptides.

### SUMMARY OF THE INVENTION

The present invention provides an improved method of monoclonal antibody production. Specifically, the invention provides a method of producing monoclonal antibodies against a protein of interest (“POI”) by immunizing a non-human animal with a fusion protein between a Th2 cytokine and the POI.

In one embodiment, the Th2 cytokine is selected from the group consisting of IL-4, IL-5, IL-10, IL-13 and IL-31. In a specific embodiment, the Th2 cytokine is IL-4.

The method of the present invention applies to production of monoclonal antibodies against essentially any POI of interest. In one embodiment, the POI is a cell surface receptor protein. Examples of cell surface receptor proteins include, but are not limited to, T-cell receptor chains, Toll-like receptors, CD23, NK-cell receptors, and tissue factor. In other embodiments, the POI is a soluble protein (i.e., not associated or attached to cell surface).

A fusion protein between a Th2 cytokine and a POI can be made by first creating a nucleic acid molecule encoding the fusion protein via linking the nucleic acid sequence encoding the Th2 cytokine in frame with the nucleic acid sequence encoding the POI.

In one embodiment, the Th2 cytokine is fused to the N-terminus of the POI. In another embodiment, the Th2 cytokine is fused to the C-terminus of the POI. In some embodiments, a spacer is included in the fusion protein that separates the Th2 cytokine and the POI.

The nucleic acid encoding a fusion protein can be introduced into an appropriate host cell for recombinant expression. The host cell can be selected from bacterial, yeast, insect or mammalian cells. The fusion protein so expressed can be isolated from the host cell or culture media and used for immunization of a non-human animal and subsequent generation of hybridomas that produce monoclonal antibodies.

The present invention also provides related compositions, including fusion proteins, expression vectors, monoclonal antibodies produced, and kits for practicing the method of the present invention.

### BRIEF DESCRIPTION OF THE DRAWINGS

FIG. 1: Boosting effect of IL-4/POI on the immune response by bridging of the B-cell receptor (BCR) and the IL-4 receptor (IL-4R).

FIG. 2: The IL-4/POI expression system. (A) IL-4 expression cassette to be incorporated in an expression vector. (B) Example of a multiple cloning site (MCS) for cloning of the POI. (C) Map of the recombinant IL-4/POI. The leader (L) peptide is removed during intracellular processing.

FIG. 3: SDS-PAGE of recombinant IL-4/TCR fusion protein (left panel) and purified TCR protein after enzymatic digestion of the tag (right panel). Both proteins were expressed under similar conditions in CHO cells using either the IL-4 expression system (left panel) or an IgG fusion protein expression system (right panel) based on the same vector backbone. The arrows point to the recombinant proteins. The left lanes of both images show molecular weight markers.

FIG. 4: Expression of genes with the IL-4/POI system increases protein expression. Mammalian cells were transfected with different plasmid constructs to express a ‘difficult to express’ toll like receptor (TLR) protein. The left image shows the control (no gene/no protein expression). The image in the middle was obtained after transfection of the cells with a commercial vector using a common His₆/myc tag linked to the TLR. The right panel shows the expression of the TLR gene as an IL-4 tagged fusion protein.

FIG. 5: IL-4/POI are expressed in high concentrations and promote the secretion of the recombinant protein. Cells were transfected with plasmid constructs to express tissue factor (TF) protein as an immunoglobulin (Ig) or an IL-4 fusion protein. The left image shows expression of the Ig tagged TF protein and the image in the middle IL-4/TF protein. The graph at the right site shows the secreted TF fusion proteins using both expression systems indicating a high increase in secretion for IL-4/TF.

FIG. 6: IL-4/POI share high structural similarity to the native proteins and monoclonal antibody (mab) development to the native proteins is supported by IL-4 tagged proteins. T-cell receptors (TCR) were expressed as IL-4 fusion proteins, purified and used for immunization of mice. Mabs from the resulting cell fusions detected the TCRγ protein on peripheral blood cells (right image). The procedure was previously performed with two different Ig/TCR fusion proteins and did not result in successful mab development using the Ig fusion proteins.

FIG. 7: IL-4/CD23 almost exclusively results in mabs that detect the native CD23 protein on IgM+B-cells by flow cytometric analysis. As expected, T-cells (CD4 or CD8 positive) or monocytes (CD14+) did not express high levels of CD23.

### DETAILED DESCRIPTION OF THE INVENTION

It has been identified in accordance with the present invention that the success rate of production of monoclonal antibodies against a POI can be significantly improved by immunization of an animal using a fusion protein formed between the POI and a Th2 cytokine. For example, a POI can be fused to a Th2 cytokine such as IL-4, and the fusion protein can be recombinantly expressed and used for monoclonal antibody production against the POI in non-human animals. Accordingly, the present invention provides a method of producing monoclonal antibodies against a POI by immunizing a non-human animal a fusion protein formed between a Th2 cytokine and the POI. The present invention also provides related compositions, including fusion proteins, expression vectors, and monoclonal antibodies produced.

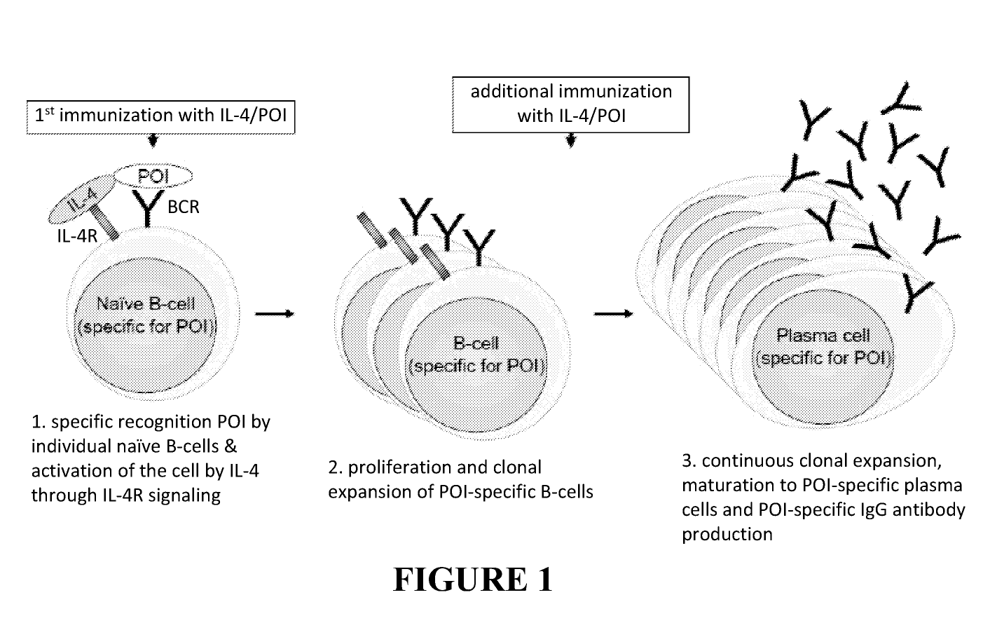

Without limiting to any particular theory, it is believed that a Th2 cytokine in the fusion protein directly and selectively influences the development of POI-specific B-cell clones. For example, the boosting effect of IL-4/POI is believed to result from a targeted stimulation of exactly those naïve B-cells that recognize the POI, and IL-4/POI is able to bridge the B-cell receptor (BCR) and the IL-4 receptor (IL-4R) expressed on naïve B-cells (FIG. 1). Thus, the fusion protein stimulates the maturation and antibody production by those B-cell clones that specifically recognize the POI. In addition, IL-4 induces class switching in these B-cells and thus enhances the development of IgG producing POI-specific plasma cells.

The monoclonal antibody production method of the present invention provides a number of advantages over the existing methodologies. Even though IL-4 is also produced during immunization with immunogenic POI by specific T-helper cells which activate the POI-specific B-cells resulting in antibody production, a number of POIs do not induce this mechanism effectively. This is likely due to a lack of efficient T-cell epitopes or structural modifications during the recombinant expression and purification process of these POIs. The IL-4/POI replaces, at least partially, the role of the T-helper cells during B-cell development. IL-4/POI supports the B-cell maturation and antibody production process. In addition, the inclusion of a Th2 cytokine such as IL-4 at the N-terminus of the fusion protein facilitates the secretion and purification of those POIs that are difficult to express using other expression systems.

Th2 Cytokines

As used herein, “Th2 cytokines” refer to cytokines secreted by T helper 2 (Th2) cells, including, for example, IL-4, IL-5, IL-10, IL-13 and IL-31. Th2 cytokines act to stimulate B cells to produce antibody by binding to specific receptors on the B cells.

For purposes of making a fusion protein, it is not necessary to use the Th2 cytokine from the same species which is to be immunized with the fusion protein. In other words, for immunization of a species with a fusion protein, the Th2 cytokine in the fusion protein can be derived from the same species or a different species. Th2 cytokines from different species share significant homologies and are believed to function effectively across species. For example, the examples hereinbelow have shown that a fusion protein between equine IL-4 and a POI effectively enhanced monoclonal antibody production in mice. Accordingly, Th2 cytokines suitable for use in the present invention can be of any animal species that express Th2 cytokines, including mammalian species, e.g., human, rodent (including mouse and rat), feline, canine, equine, bovine, ovine, caprine, porcine, and monkey; as well as avian (e.g., chicken), marine, amphibian species, among others. The protein and nucleic acid sequences for Th2 cytokines have been identified from a variety of animal species and are available through, e.g., GenBank database. As illustration, SEQ ID NOS: 2, 4 and 6 set forth the protein sequences of full-length human, murine and equine IL-4.

Nucleic acid sequences encoding either the full-length form or the mature form (i.e., the full-length minus the signal or leader peptide) of a Th2 cytokine can be used in making a fusion protein. In some embodiments, naturally-occurring (wild type) Th2 cytokines are used in making fusion proteins. It should be recognized that allelic variations may exist for a Th2 cytokine within an animal species; i.e., there might be several naturally-occurring (wild type) allelic sequences for a Th2 cytokine, all of which are suitable for use in making the fusion protein of the invention. In other embodiments, mutant or genetically modified forms of Th2 cytokines are used as long as the mutant forms substantially retain the activity (e.g., binding to receptor, and/or stimulation of B cells) of the wild type cytokine. By “substantially” is meant at least 75%, 80%, 85%, 90%, 95% or greater.

POI

The monoclonal antibody production method of the present invention is essentially applicable to generating monoclonal antibodies against any protein of interest (“POI”). POIs of the present invention do not include, however, epitope or protein tags routinely used in recombinant expression, purification and/or detection of proteins such as a His tag, Myc, flag, HA and green fluorescence protein (GFP).

In one embodiment, the POI is a peptide or polypeptide of at least 7 or 8 amino acids, or at least 10, 15, 20, 25, 30, 40, 50, 75, 100, 125, 150, 175, 200, 225, 250, 275 or 300 amino acids or more.

In another embodiment, the POI is a cell surface receptor protein. By “cell surface receptor protein” is meant a protein that is localized on the cell surface, typically a membrane protein having one or more transmembrane segments, and binds a ligand outside of the cell thereby triggering a signaling cascade inside the cell. Examples of cell surface receptor proteins include, but are not limited to, T-cell receptor (“TCR”) chains (such as, e.g., TRAC (T-cell receptor alpha constant region), and TRBC (T-cell receptor beta constant region)), Toll-like receptors, CD23, CD25, CD28, CD16, IL-4Rα (interleukin-4 receptor cc), NK-cell receptors (such as NKp46, also known as natural killer cell protein 46 or natural killer cell receptor 1), and tissue factor, some of which are difficult to express using other recombinant expression systems and also difficult to produce monoclonal antibodies against.

T cell receptor is a heterodimeric molecule on the surface of T cells that consists of an α and β chain, or less frequently a γ and δ chain. TCR is a member of the immunoglobulin superfamily and its structure is characterized by an N-terminal immunoglobulin (Ig)-variable (V) domain, an Ig-constant (C) domain, a transmembrane/cell membrane-spanning region, and a short cytoplasmic tail at the C-terminal end. TCR chains were previously reported to be difficult to express in their native form when prokaryotic systems were used (Novotny et al. 1991, Ward 1992, Hoo et al. 1992). In previously reported mammalian systems, the successful expression of TCR chains required rather complicated expression strategies (Slanetz and Bothwell 1991, Engel et al. 1992, Weber et al. 1992, Callan et al. 1993, Chang et al. 1994). In contrast, the IL-4 fusion protein system of the present invention provides a simple and effective expression system for secreted TCR chains.

Toll-like receptors (TLRs) belong to a group of receptor proteins named “pattern recognition receptors” that recognize structurally conserved molecules derived from microbial pathogens (or “pathogen-associated molecular patterns (PAMPs)”) and activate immune responses upon recognition of these microbial molecules. TLRs are single membrane-spanning non-catalytic proteins and together with the IL-1 receptors form the “Interleukin-1 Receptor/Toll-Like Receptor Superfamily”. All members of this family have in common a so-called TIR (Toll-IL-1 receptor) domain. Known mammalian TLRs include at least TLR-1, TLR-2, TLR-3, TLR-4, TLR-5, TLR-6, TLR-7, TLR-8, TLR-9, TLR-10, TLR-11, TLR-12, TLR-13, TLR-14 and TLR-15, all of which are contemplated by the present invention.

CD23, also known as FcεRII, is the “low affinity” receptor for IgE, an antibody isotype involved in allergy and resistance to parasites. There are two forms of CD23: CD23a and CD23b. CD23a is expressed on follicular B cells, while CD23b requires IL-4 to be expressed on T-cells, monocytes, Langerhans cells, eosinophils and macrophages. Both forms of CD23 are suitable for use as a POI in the present invention.

NK-cell receptors are molecules of the killer cell inhibitory receptor (KIR) or Ly49 families. These receptors are characteristic for natural killer (NK) cells. They represent a variety of major histocompatibility complex (MHC)-specific receptor molecules which share the common function of silencing NK cells through “self” MHC recognition. This is to avoid cytotoxic NK cells from attacking the host's “self” tissues. However, in case of infection with foreign pathogens, the inhibitory effects of KIR and/or Ly49 receptors can quickly be overcome by activating signals through other membrane receptors on these cells, such as CD16, CD56 or other activating receptors. This principle of balancing inhibitory and activating signals makes NK-cells very potent in the rapid killing of pathogens.

Tissue factor (“TF”), also known as factor III, thrombokinase, or CD142, is the cell surface receptor for the serine protease factor VIIa. TF is expressed in subendothelial tissue, platelets, and leukocytes. Once bound to factor VIIa, the complex of TF with factor VIIa catalyzes the conversion of the inactive protease factor X into the active protease factor Xa, a step in the coagulation cascade. TF has 3 distinct domains: extracellular (factor VIIa binding), transmembrane, and cytoplasmic (signal transduction).

In still another embodiment, the POI is a soluble protein, i.e., a protein not associated with or attached to cell surface. Examples of soluble proteins include naturally-occurring cytokines, such as IL-8, interferons (such as IFN-β), CXCL9, CXCL10, IL-13, IL-22BP (interleukin 22 binding protein), IL-1β, and IL-1RA (interleukin-1 receptor antagonist), for example; as well as extracellular regions of naturally-occurring cell surface proteins such as CD25, CD28, CD16, IL-4Rα, NKp46, TRAC, and TRBC.

Fusion Protein

A fusion protein between a Th2 cytokine and a POI can be made by creating a nucleic acid molecule encoding the fusion protein and expressing the fusion protein from such nucleic acid in a recombinant expression system. The nucleic acid molecule encoding the fusion can be generated by linking the nucleic acid sequence encoding the Th2 cytokine in frame with the nucleic acid sequence encoding the POI.

In one embodiment, the Th2 cytokine is fused to the N-terminus of the POI. In this orientation, the Th2 cytokine can be used as an N-terminal tag for detection and purification of the fusion protein. In addition, when the full length cytokine coding sequence is used, the leader sequence (secretory signal peptide) of the cytokine can facilitate the secretion of the cytokine-POI fusion protein. Alternatively, other appropriate leader sequences, suitable for guiding the cytokine/POI fusion protein to the ER and the secretory pathway in the host cell, can be used instead of the leader sequence of the Th2 cytokine and linked to the mature sequence of the Th2 cytokine.

In another embodiment, the Th2 cytokine is fused to the C-terminus of the POI. In making a fusion of this orientation, preferably the mature form of the Th2 cytokine, rather than the full-length sequence including the leader sequence, is used. The fusion protein can rely on the leader sequence of the POI if present, or a heterologous leader sequence (from a protein other than the POI) functional in the host cell, to achieve secretion of the fusion protein.

In still another embodiment, a spacer can be incorporated between the Th2 cytokine and the POI. By “spacer” is meant a short peptide sequence that joins the Th2 cytokine and the POI, yet preserves some distance between the two proteins such that both the cytokine and POI can properly fold independently. Generally, the spacer consists of between 2 or 3 amino acids to 50 amino acids, typically between 3 to 25, or 3 to 20, or 3 to 15 amino acids. In a specific embodiment, the space consists of 3-10 amino acids. Although there is no specific restriction on the selection of amino acids for the spacer region, the amino acids can be selected to accommodate the folding, net charge, hydrophobicity or other properties of the fusion protein. Typical amino acids for use in a spacer region include Gly, Ala, Ser, Thr and Asp.

In one specific embodiment, the spacer region includes a cleavage site recognized by a protease. If desirable, the Th2 cytokine and the POI can be separated from each other by protease cleavage after recombinant expression and purification. An example of a cleavage site is the enterokinase (EK) cleavage site, (Asp)₄-Lys (SEQ ID NO: 7).

In another embodiment, the nucleotide sequence encoding the spacer is designed to include one or more endonuclease restriction sites which facilitate the formation and cloning of the fusion protein.

One of skill would recognize that modifications can be made to a Th2 cytokine, a POI or the fusion protein without diminishing their biological activities. Some modifications may be made to facilitate the cloning, expression, or incorporation of the constituent molecules into a fusion protein. For example, amino acids can be placed on either terminus to create conveniently located restriction sites or termination codons; and a methionine can be added at the amino terminus to provide an initiation site.

Recombinant Expression of Fusion Protein

For recombinant expression of a fusion protein, a nucleic acid molecule encoding the fusion protein is generally placed in an expression vector in an operable linkage to a promoter (such as the T7, trp, or lambda promoters for expression in bacteria, or a CMV promoter for expression in mammalian cells) and a 3′ transcription termination sequence, and optionally additional suitable transcriptional and/or translational regulatory elements such as a transcription enhancer sequence and a sequence encoding suitable mRNA ribosomal binding sites. Additional sequences that can be included in the expression vector include an origin of replication, and a selection marker gene to facilitate identification of transformants such as genes conferring resistance to antibiotics (e.g., the amp, kana, gpt, neo, and hyg genes).

Host cells suitable for use in the recombinant expression of the fusion protein include bacterial cells such as E. coli, and eukaryotic cells including but not limited to yeast, insect cells (e.g. SF9 cells), and mammalian cells such COS, CHO and HeLa cells.

The expression vectors can be introduced into a host cell by well-known methods such as calcium chloride transformation for bacterial cells, and calcium phosphate treatment or electroporation for mammalian cells. Cells transformed by the expression vectors can be selected based on the phenotype provided by the selectable marker gene.

Once expressed, the recombinant fusion proteins can be purified according to standard methods available in the art, such as ammonium sulfate precipitation, affinity columns, chromatography, gel electrophoresis, among others. In one embodiment, the fusion protein is purified based on affinity chromatography using antibodies specific for the Th2 cytokine component in the fusion protein. While it is preferred that the fusion protein be subjected to a purification procedure such that a partially or highly purified preparation of the fusion protein is used for immunization, a crude preparation of the fusion protein can also be used in the immunization and support mab production when used at a higher amount of total protein. By “partially purified” is meant that the fusion protein constitutes at least 50%, 60% or 70% of the total proteins in a protein preparation. Purities such as 75%, 80%, 90%, 95% or higher can be achieved and are preferred. By using affinity columns, the purity is usually at least 90%.

Production of Monoclonal Antibodies

For monoclonal antibody production, a recombinant fusion protein is used to immunize a non-human animal such as a mammalian animal, e.g., mouse, rat, rabbit, goat; an avian animal, e.g., chicken; or any other appropriate host animal, according to immunization protocols previously described (Wagner et al. 2003). The immunization generally includes two or more administrations (e.g., injections), more typically an initial administration followed by several, i.e., 3, 4, 5, 6 or 7, booster administrations during a span of about 25-35 days. Generally, to immunize an animal such as mouse, rat, rabbit and chicken, 2-50 μg of purified or partially purified fusion protein can be used in the first administration, and 1-25 μg of fusion protein is used in the booster administrations. For larger hosts such as goats, the amount of a fusion protein used in the immunization maybe 2-3 fold or more higher than an animal such as mouse. A fusion protein can be administered together with an adjuvant, such as Gerbu Adjuvant, Freund's adjuvant, among others.

Cells from the spleen (in the case of rodents, for example) or lymph nodes (in cases of larger animals) or PBMCs (in cases of larger animals), which contain antibody producing cells, are taken from the immunized animal, approximately 25-35 days, or 28-33 days, or 29, 30, 31, 32 days or even later, after the first immunization. These cells are fused to an immortalized cell line (e.g. SP2/0 or X63-Ag8.653) using a suitable fusing agent, such as polyethylene glycol, to form a hybridoma cell (see, e.g., Kearney et al. 1979). Immortalized cell lines are typically transformed mammalian cells, such as B-cell myeloma cells of rodent, bovine and human origin. In a specific embodiment, mouse myeloma cell lines are employed.

After the fusion, the cells are plated into multi-well plates, and cultured in a suitable culture medium that preferably contains one or more substances that inhibit the growth or survival of the unfused, immortalized cells. An example of a culture medium for hybridomas is the “HAT medium”, which includes hypoxanthine, aminopterin, and thymidine—these are substances that prevent the growth of HGPRT-deficient cells. After 2-3 weeks, supernatants are screened for antibodies to a POI. Cells from positive wells are then subjected to limiting dilution for isolation of single clones.

The screening of antibodies can be performed in various ways. First, antibodies specific for a POI can be identified by ELISA using the cytokine/POI fusion protein directly for coating or indirectly via anti-cytokine coating followed by an incubation step with the cytokine/POI fusion protein. Second, by using a transient or stable transfectant expressing the cytokine/POI fusion protein, one can identify antibodies recognizing IL-4/POI based on intracellular staining. IL-4 specific antibodies can be identified and excluded by the using recombinant IL-4 by ELISA or IL-4 transfectants without the POI for intracellular staining. Third, POI-specific antibodies can be identified directly on target cells expressing native POI or solutions containing native POI. Additional screening methods such as Western blotting or immunohistochemistry can be used to further identify appropriate antibodies for specific procedures.

Monoclonal antibodies produced in accordance with the present invention include all classes of immunoglobulins, i.e., IgG, IgM, IgE, IgA, IgD, and any subclass thereof. As a result of the B-cell stimulatory function of a Th2 cytokine, the method of the present invention provides a significantly improved production of monoclonal antibodies, relative to a method using only the POI without a Th2 cytokine. The improvement is reflected by an increased number of positive hybridoma clones, increased likelihood of obtaining antibodies with varying affinities including higher affinities to POI, and increased likelihood of obtaining antibodies of different classes including particularly IgG subclasses.

Kit for Production of Monoclonal Antibodies

The invention also provides a kit for custom production of monoclonal antibodies against a POI. The kit includes an expression vector, containing a promoter, a Th2 cytokine coding sequence, a cloning site located at or near the C-terminus of the cytokine coding sequence for convenient insertion of a nucleic acid coding for a POI to form cytokine/POI fusion, and a 3′ transcription termination sequence. The kit also contains a host cell, reagents for purification of the recombinant fusion protein, adjuvant for immunization, immunization instructions, and reagents for screening for and identification of POI-specific monoclonal antibodies.

### Example-1

This Example describes the general methods used in the experiments described in Examples 2-5.

Generation of the IL-4 Expression Vector

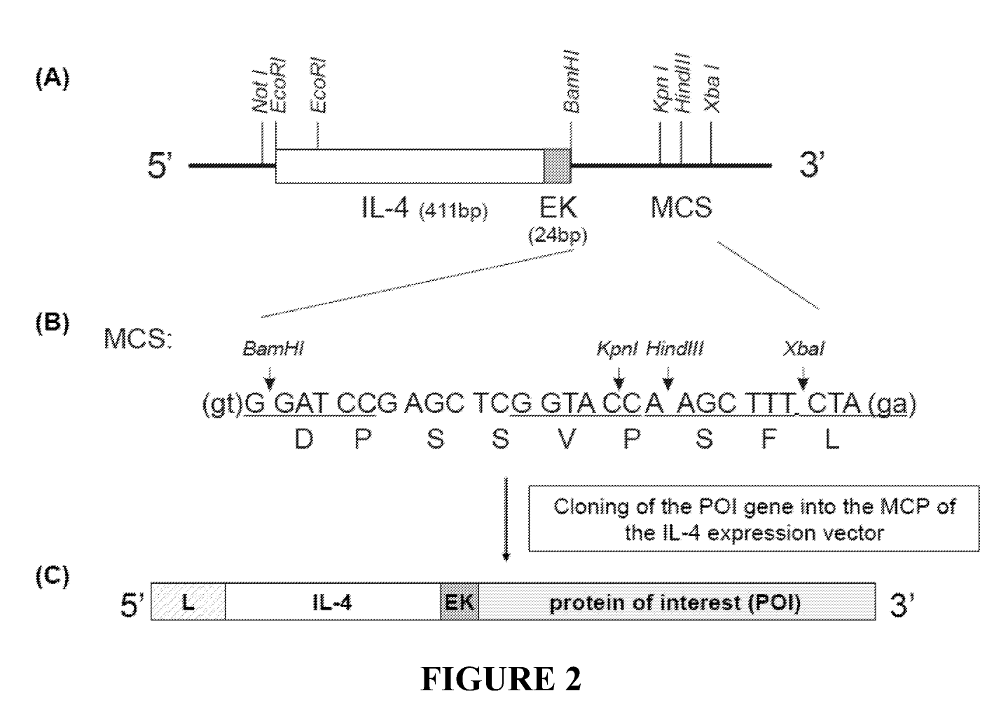

A complete equine IL-4 cDNA including the leader sequence was amplified by PCR using the forward (5′ GCGGCCGCATGGGTCTCACCTACCAACTG 3′, SEQ ID NO: 8) and reverse (5′ CGTCGTACAGATCACACTTGGAGTATTTCTCTTTC 3′, SEQ ID NO: 9) primers. A complete gene sequence encoding an enterokinase (EK) cleavage site, (Asp)₄-Lys (SEQ ID NO: 7) (Anderson et al., 1977), was designed at the 3′ end of the IL-4 by an additional PCR using the same forward primer and an EK reverse primer (5′ CCGGATCCTTATCGTCATCGTCGTACAGATC 3′, SEQ ID NO: 10). The amplification resulted in an IL-4/EK cDNA fragment. The IL-4 forward primer included a NotI site (5′) and the EK reverse primer included a BamHI site (3′) for subsequent cloning of the IL-4/EK cDNA into the multiple cloning site of a mammalian expression vector with a CMV promoter. A multiple cloning site at the 3′ end of the IL-4/EK gene was used to clone a gene of interest into the expression vector (FIG. 2A).

Cloning of POI in the IL-4 Expression Vector

The cDNA of the (POI) was amplified by PCR using POI gene specific primers. The 5′ primer included a restriction site (e.g. BamHI) for in-frame cloning of the POI cDNA with the IL-4/EK gene into the expression vector. The reverse primer contained a restriction site compatible with the multiple cloning site (e.g. HindIII or KpnI) and also a stop codon at the 3′ end of the POI gene sequence. After amplification the POI cDNA was cloned into the multiple cloning site of the IL-4 expression vector. All DNA cloning steps were controlled by nucleotide sequencing.

IL-4/POI Expression

The IL-4/POI expression vector was then used to transfect Chinese Hamster Ovary (CHO) cells. To obtain stable transfectants, a total of 8-12 μg DNA of the IL-4/POI expression vector was linearized with PvuI and purified by phenol extraction and ethanol precipitation (Sambrook et al., 1989). The transfection of the cells was performed using Geneporter II Transfection Reagent (Gene Therapy Systems, San Diego, Calif., USA). The following day, the expression of IL-4/POI was detected within the cells and in the cell culture supernatant by flow cytometry and ELISA, respectively. The transfected cells were subsequently plated into 96 well plates using 100 cells/well in F12 medium (Invitrogen, Carlsbad, Calif., USA), containing 10% (v/v) FCS (Hyclone, Logan, Utah, USA), 50 μg/ml gentamycin, and 1.5 mg/ml geneticin (G418) (both from: Invitrogen, Carlsbad, Calif., USA). After G418 selection, clones were selected for highest secretion of the fusion protein by ELISA and cloned by limiting dilution until the entire cell population expressed the fusion protein as measured by intracellular staining. To collect serum free supernatants, stable transfectants were grown until they were 70-80% confluent. Then, the cells were washed with F12 medium without FCS and maintained for 2-4 days in F12 medium with 50 μg/ml gentamycin.

ELISA for IL-4/POI

To detect equine IL-4/POI, an ELISA for IL-4 was used. All ELISA buffers, washing steps, the substrate solution and the measurement of the assay were the same as described previously in detail (Wagner et al., 2003). For the IL-4 ELISA, an affinity purified monoclonal anti-IL-4 antibody (Wagner et al. 2006) was coated to the ELISA plates in a concentration 5 μg/ml and incubated overnight at 4° C. After washing the plates, the cell culture supernatants from the transfectants were applied. For quantification, purified recombinant IL-4 was added in two-fold dilutions ranging from around 1 μg/ml to 62.5 ng/ml. The purified IL-4 used was obtained by enterokinase digestion of recombinant IL-4/IgG1 (Wagner et al. 2005). Samples and standards were incubated for 1-2 hours at room temperature. For detection a polyclonal anti-equine IL-4 antiserum or second monoclonal anti-IL-4 antibody was used. This antibody was directly or indirectly (via biotin) conjugated to an enzyme, e.g. horseradish-peroxidase. The incubation with the biotin conjugated anti-equine IL-4 was followed by an additional streptavidin-peroxidase incubation (Jackson ImmunoResearch, West Grove, Pa.) and substrate solution.

IL-4/POI Purification

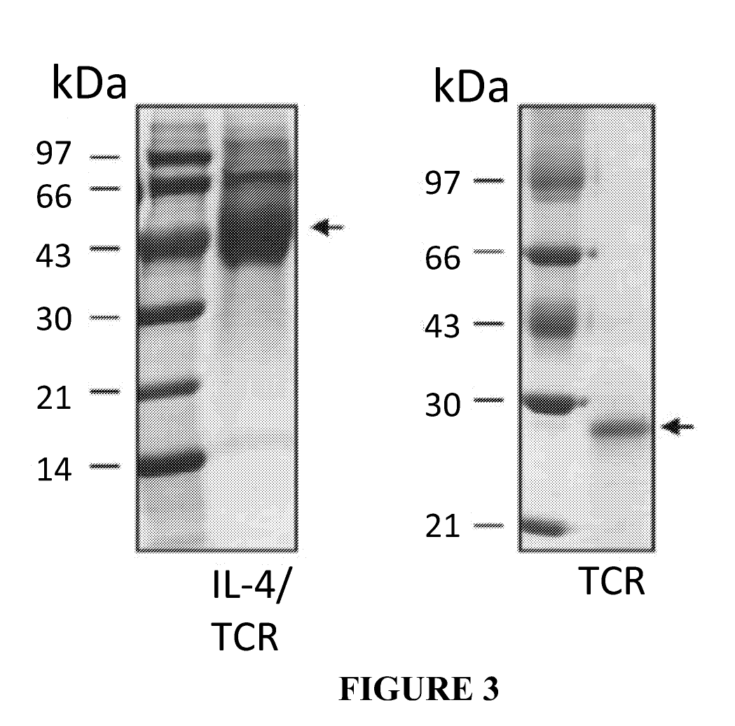

The IL-4/POI protein was purified from the supernatant using an IL-4 affinity column (FIG. 3, left panel). The affinity column was made by coupling a monoclonal anti-equine IL-4 antibody (Wagner et al. 2006) to CNBr-activated sepharose according to standard protocols. The IL-4/POI was eluted from the column using 0.1M glycine pH 3.0 and the eluate was subsequently neutralized with 1M Tris pH 8.0. The IL-4/POI was dialyzed against a physiological solution such as PBS or 0.9% NaCl and concentrated using a filter concentrator of appropriate molecular cut-off (e.g. Millipore).

Antibody Production and Screening

The complete recombinant IL-4/POI was then used to immunize mice or other mammals for monoclonal antibody production essentially according to protocols previously described (Wagner et al. 2003). Briefly, mice were immunized on days 0, 14, 21, 28, 29 and 30, using 2-50 μg purified IL-4/POI for the first injection and 1-25 μg IL-4/POI for all following booster injections. Serum was taken before the first, second and third immunization and on the day of the fusion. All sera are tested by ELISA. Spleen cells were taken from the mice on day 31, fused to X63-Ag8.653 myeloma cells (Kearney et al. 1979), and then plated into 24 well plates with 2 ml cell suspension per well. After 2-3 weeks, supernatants were tested by ELISA for antibodies to POI. Single cell clones were picked from positive wells and transferred into individual wells of fresh 24 well plates. Cells were cultured for an additional 3-5 days before the supernatants were tested for antibodies to POI by ELISA. Positive supernatants were identified and subsequently tested in parallel for recognition of rIL-4. Antibodies which detected the POI, but not the rIL-4, were considered as potentially POI specific and were further characterized. Cell cultures were tested for clonality by murine isotype ELISA (Sigma, St. Louis, Mo.) and flow cytometric analysis using a FITC conjugated goat anti-mouse IgG(H+L) antibody (Jackson ImmunoResearch Lab., West Grove, Pa.). Cultures were cloned by limiting dilution until they secreted a single mouse isotype and showed a uniform Ig positive population by intracellular staining with anti-mouse Ig.

### Example-2

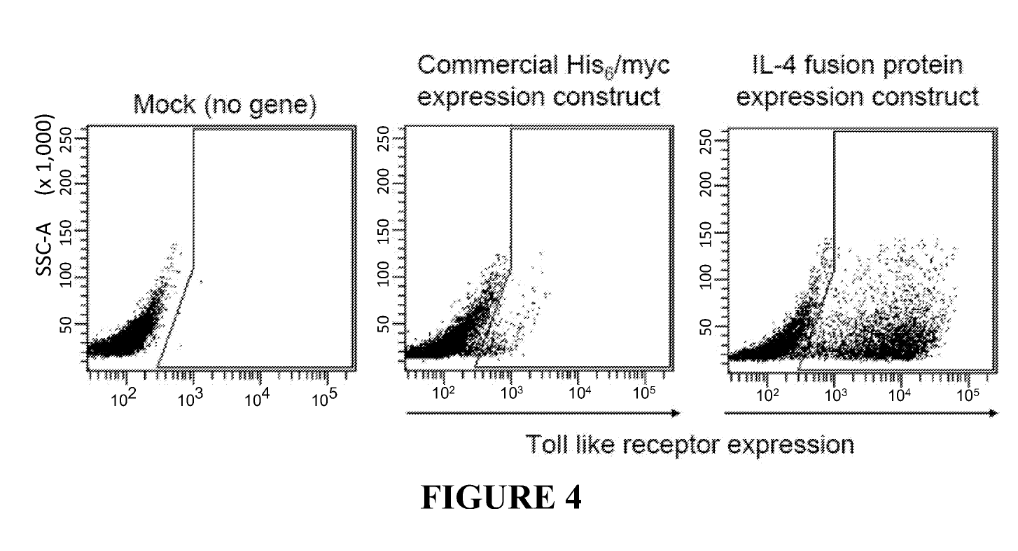

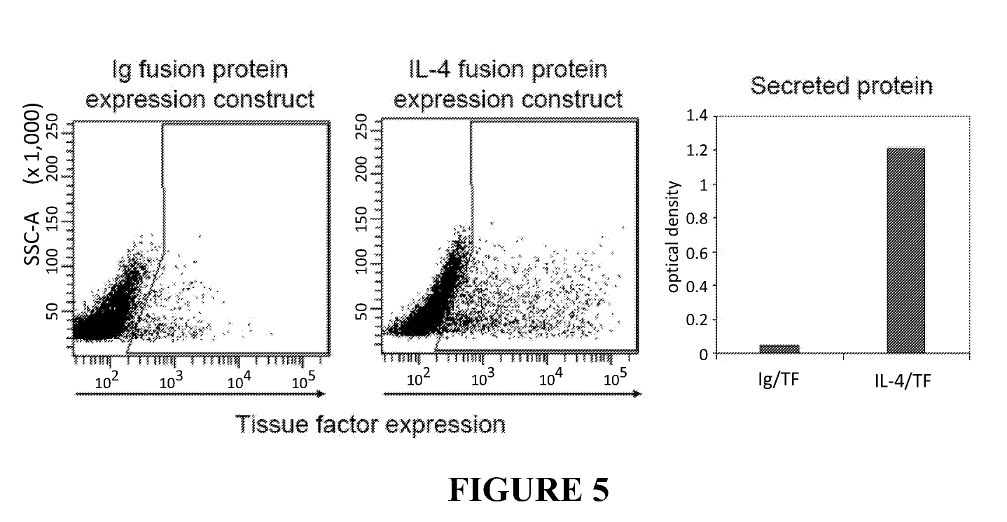

This example shows that the new IL-4/POI expression system resulted in increased expression of the recombinant POI. IL-4/POI and other available expression system are compared in FIG. 4 for POI being a Toll-like receptor (TLR) protein (SEQ ID NO: 11) and in FIG. 5 for POI being a tissue factor (TF) cytokine (SEQ ID NO: 12).

### Example-3

Example 3 shows that the secretion of the POI was enhanced by using the new IL-4/POI system compared to expressing the same protein (here TF) in another available expression system (FIG. 5). Secretion of TLR2 (toll-like receptor 2) was also shown to be enhanced through the use of the IL-4/POI system. IL-4 is a secreted protein. The use of IL-4 and its leader peptide contributed to the secretion of the entire IL-4/POI, even if the POI itself is not a secreted protein. In contrast, neither TF nor TLR2 was secreted by using an IgG fusion or an His-myc tag. Secretion of the POI is preferred during expression because the POI is easier to purify from supernatant and its structural confirmation is less modified because of the simplified purification process compared to the purification of intracellular proteins.

### Example-4

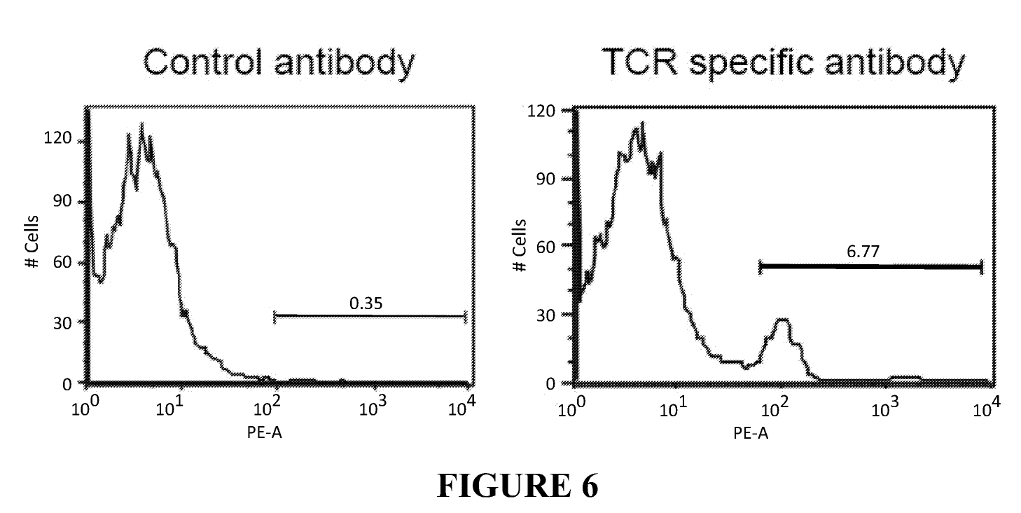

This example shows that by using the new IL-4/POI system, antibodies to native proteins were obtained. In the example the POI was a T-cell receptor (TCR) protein (SEQ ID NO: 13). When IL-4/TCR was used for immunization antibody development to native TCR was successful (FIG. 6), while antibodies to the native TCR protein were not obtained by using the isolated TCR without the IL-4 tag (FIG. 3, right panel)

### Example-5

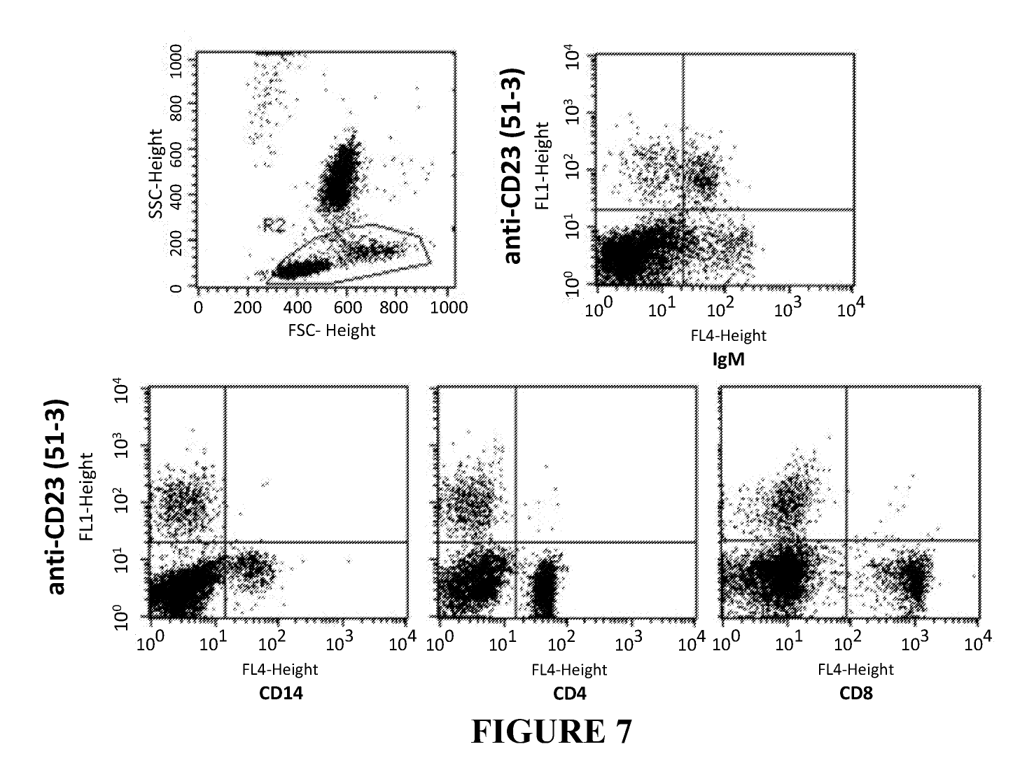

Example 5 shows the boosting effect of the IL-4/POI on monoclonal antibody production. In the example the POI was CD23. The use of IL-4/CD23 resulted in an approximately 10-fold higher number of positive clones to CD23 than commonly seen in hybridoma technology (Table 1). The direct comparison of positive anti-IL-4 and anti-CD23 positive clones in the table underlines this increase. While the mouse responded as expected to the IL-4 and average number of anti-IL-4 clones was obtained, the number of anti-CD23 clones from the same mouse was significantly increased (6-fold more anti-CD23 clones than anti-IL-4 clones in the same fusion). Out of the initially positive anti-CD23 clones, 26 were tested in more detail. Out of these, 19 stable clones survived the selection and cell culture process for monoclonal antibody development and all 19 detected the native CD23 on B-cells (FIG. 7).

TABLE 1
IL-4/POI boosts the immune response and results
in a highly increased number of monoclonal antibody
clones to CD23 and only few clones to IL-4.

| | Target | initial screenings; # positives | | |
|---|---|---|---|---|
| Species | molecule | ELISA | flow cytometry | both |
| Horse | IL-4/CD23 | 160 (480 wells) | 19* | 19* |
| Horse | IL-4/CD23 | 42 (480 wells) | 5⁺ | 5⁺ |

*best 26 clones tested
⁺best 12 clones tested

### Example-6

This example shows that the yields of proteins produced from the IL-4/POI fusion system disclosed herein exceeded yields obtained from other fusion systems.

Table 2 shows a comparison of total protein amounts that were obtained with the IL-4 fusion protein system and an IgG fusion protein system (the latter system has been described in Wagner et al. 2005, 2008). When previously used in mammalian cells, the IgG system was found to give higher yields of better purity than conventional systems using His-tags (Dohmann et al. 2000; Wagner et al. 2005). The IL-4 and IgG fusion protein systems were then compared in the same cell line and by using the same transfection and selection procedures. One liter of cell culture supernatant from each stable transfectant was used for purification of different recombinant proteins. The mean protein yield obtained from the IgG system was 0.232 mg. The IL-4 fusion protein system resulted in a mean recombinant protein yield of 1.34 mg per liter culture supernatant.

TABLE 2
Comparison of protein yields obtained from 1 liter of stable transfectant
culture supernatant with IL-4 and IgG fusion protein systems.

| Fusion protein | | Protein | Mean |
|---|---|---|---|
| system | Protein | yield (mg) | yield (mg) |
| IgG | IFN-b | 0.30 | 0.232 |
| | CXCL9 | 0.13 | |
| | CD25 | 0.17 | |
| | CD28 | 0.14 | |
| | IL-4Ra | 0.25 | |
| | IL-13 | 0.40 | |
| IL-4 | NKp46/IL-4 | 1.95 | 1.34 |
| | CD16/IL-4 | 0.45 | |
| | IL22BP/IL4 | 1.00 | |
| | CD23/IL-4 | 2.30 | |
| | canTF/IL-4 | 1.00 | |

Because much higher total yields were obtained with the IL-4 fusion proteins, the purification procedure was also adjusted to a small scale method. Small scale purifications were done from 160-200 ml of supernatant from transient transfections. The advantage is that the small scale procedure saves production time on the transfectant part and the proteins can be directly used to start the immunization of mice (i.e. almost immediately after the expression vector was produced). The supernatant from the transient transfection can be harvested 2-3 days after the transfection, while the establishment and selection of a high yield stable transfectant can take 6-12 weeks. Table 3 shows the protein yield obtained from the small scale procedure using IL-4 fusion proteins. The procedure was also tested on IgG fusion proteins which resulted in suboptimal to almost undetectable protein amounts and was not continued. For IL-4 fusion proteins the mean protein concentration was 306 μg/ml. For comparison with the stable transfection methods the protein yield was calculated for 1 liter of transient transfection supernatant resulting in a mean yield of 0.459 mg for the transient transfection method using IL-4 fusion proteins. This almost doubled the yield of IgG fusion proteins obtained by stable transfection.

TABLE 3
Protein yields obtained from small scale transient tranfections
(160-200 ml cell culture supernatant) using IL-4 fusion proteins.

| | Protein concentration | Calculated yield | Mean |
|---|---|---|---|
| | purified IL-4 fusion protein | (μg) per liter | yield |
| protein | in 300 μl (μg/ml) | supernatant* | (mg) |
| eqTF/IL-4 | 280 | 420 | 0.459 |
| CXCL9/IL-4 | 350 | 525 | |
| IL-1b/IL-4 | 200 | 300 | |
| IFN-b/IL-4 | 300 | 450 | |
| CXCL10/IL-4 | 150 | 225 | |
| IL-1RA/IL-4 | 300 | 450 | |
| TRAC/IL-4 | 210 | 315 | |
| TRBC/IL-4 | 660 | 990 | |

*(conc. μg/ml × 3/10 × 5)

### Example-7

This example further demonstrates mAb production using IL-4 fusion proteins. Various proteins were used for mAb production. Four of these fusions have been tested, two have been completed. All fusions resulted in a high number of positive clones, mAbs of different epitope specificities (likely to work in ELISA or have different functional properties such as stimulation or inhibition), and mAbs that can be used in various applications (Table 4).

A notable example is CXCL9. This is a small chemokine of low immunogenicity as shown in two unsuccessful fusions using yeast expressed CXCL9. These two yeast protein fusions were performed by us using the same method as for the CXCL9/IL-4 fusion. The yeast fusions did not result in any CXCL9 specific mAbs. The single CXCL4/IL-4 fusion resulted in 10 CXCL9 positive clones. This direct comparison showed that the IL-4 fusion protein boost the immune response during immunization and can result in successful mAb production for proteins that fail if expressed in other systems. It also supports the conclusion that the IL-4 fusion protein can overcome limitations in mAb development for small proteins of low immunogenicity.

TABLE 4
mAb production using IL-4 fusion proteins

| IL-4 | Total clones | | mAbs with | |
|---|---|---|---|---|
| fusion | (indicator for | | different | |
| protein | proliferating | Positive | speci- | |
| partner | B-cells) | clones | ficities | Applications |
| CD23 | >2000 | >50% pos | >7 | Flow cytometry, |
| | | clones, only 26 | | Western blotting, |
| | | selected for | | ELISA |
| | | processing | | |
| TF | >400 | 20 | >4 | ELISA, flow |
| | | | | cytometry, |
| | | | | Western blotting, |
| | | | | immunohisto- |
| | | | | chemistry |
| IFN-β | >900 | 14 | In progress | ELISA & in |
| | | | | progress |
| CXCL9 | >700 | 10 | In progress | ELISA & in |
| | | | | progress |

| SEQ | |
|---|---|
| ID NO | Description |
| 1 | human IL-4 nucleic acid sequence (NM_000589; CDS 371-832) |
| 2 | human IL-4 protein (NP_000580; the first 24 amino acids |
| | representing the signal sequence) |
| 3 | murine IL-4 nucleic acid sequence (NM_021283; CDS 60-482) |
| 4 | murine IL-4 protein (NP_067258; the first 20 amino acids |
| | representing the signal sequence) |
| 5 | equine IL-4 nucleic acid sequence (GU139701) |
| 6 | equine IL-4 protein (ACZ27799; the first 24 amino acids |
| | representing the signal sequence) |
| 7 | enterokinase cleavage site |
| 8 | forward primer for amplifying full-length equine IL-4 |
| 9 | reverse primer for amplifying full-length equine IL-4 |
| 10 | reverse primer including the enterokinase cleavage site |
| 11 | human TLR9 protein (an example of Toll like receptor) |
| 12 | equine tissue factor without leader peptide |
| 13 | bovine TRGC3/extracellular region (an example of T-cell |
| | receptor constant region) |

### REFERENCES

Anderson, L. E., Walsh, K. A. and Neurath, H., 1977. Bovine enterokinase. Purification, specificity, and some molecular properties. Biochemistry 26: 3354-3360.
Callan, M. F., Reyburn, H. T., Bowness, P., Ottenhoff, T. H., Engel, I., Klausner, R. D., Bell, J. I. and McMichael, A., 1993. A method for producing monoclonal antibodies to human T-cell-receptor beta-chain variable regions. Proc. Natl. Acad. Sci. USA. 90: 10454-10458.
Chang H C, Bao Z Z, Yao Y, Tse A G D, Goyarts E C, Madsen M, Kawasaki E, Brauer P P, Sacchettini J C, Nathenson S G, Reinherz E L. 1994. A general method for facilitiating heterodimeric pairing between two proteins: Application to expression of α and β T-cell reseptor extracellular segments. Proc. Natl. Acad. Sci. USA 91: 11408-11412.
Chomarat, P. and Banchereau, J., 1998. Interleukin-4 and interleukin-13: their similarities and discrepancies. Int. Rev. Immunol. 17: 1-52.
Dohmann K, Wagner B, Horohov DW and Leibold W. 2000. Expression and characterisation of equine interleukin 2 and interleukin 4. Vet. Immunol. Immunopathol., 77: 243-256.
Engel, I., Ottenhoff, T. H. and Klausner, R. D., 1992. High-efficiency expression and solubilization of functional T cell antigen receptor heterodimers. Science 256: 1318-1321.
Esser, C. and Radbruch, A., 1990. Immunoglobulin class switching: molecular and cellular analysis. Annu. Rev. Immunol. 8: 717-735.
Hoo, W. F., Lacy, M. J., Denzin, L. K., Voss, E. W. Jr., Hardman, K. D. and Kranz, D. M., 1992. Characterization of a single-chain T-cell receptor expressed in Escherichia coli. Proc. Natl. Acad. Sci. USA 89: 4759-4763.
Kearney, J. F., Radbruch, A., Liesegang, B., Rajewsky, K., 1979. A new mouse myeloma cell line that has lost immunoglobulin expression but permits the construction of antibody-secreting hybrid cell lines. J. Immunol. 123, 1548-1550.
Novotny, J., Ganju, R. K., Smiley, S. T., Hussey, R. E., Luther, M. A., Recny, M. A., Siliciano, R. F. and Reinherz, E. L., 1991. A soluble, single-chain T-cell receptor fragment endowed with antigen-combining properties. Proc. Natl. Acad. Sci. USA 88: 8646-8650.
Paul. W., 1987. Interleukin 4/B cell stimulatory factor 1: one lymphokine, many functions. FASEB J. 1: 456-461.
Sambrook, J., Fritsch, E. F. and Maniatis, T., 1989. Molecular cloning: a laboratory manual, Cold Spring Harbor Laboratory Press, New York.
Siebenkotten, G., Esser, C., Wabl, M. and Radbruch, A., 1992. The murine IgG1/IgE class switch program. Eur. J. Immunol. 22: 1827-1834.
Slanetz, A. E. and Bothwell, A. L., 1991. Heterodimeric, disulfide-linked alpha/beta T cell receptors in solution. Eur. J. Immunol. 21: 179-183.
Sonoda, Y., 1994. Interleukin-4: a dual regulatory factor in hematopoiesis. Leuk. Lymphoma. 14: 231-240.
Wagner, B., Radbruch, A., Rohwer, J. and Leibold, W., 2003. Monoclonal anti-equine IgE antibodies with specificity for different epitopes on the immunoglobulin heavy chain of native IgE. Vet. Immunol. Immunopathol. 92: 45-60.
Wagner, B., Robeson, J., McCracken, M., Wattrang, E. and Antczak, D. F., 2005. Horse cytokine/IgG1 fusion proteins—mammalian expression of biologically active cytokines and a system to verify antibody specificity to equine cytokines. Vet. Immunol. Immunopathol. 105: 1-14.
Wagner, B., Hillegas, J. M. and Antczak, D. F., 2006. A monoclonal antibody to equine interleukin 4. Vet. Immunol. Immunopathol. 110: 363-367.
Wagner, B., Hillegas, J. M., Brinker, D. R., Horohov, D. W., Antczak, D. F., 2008. Characterization of monoclonal antibodies to equine interleukin-10 and detection of T regulatory 1 cells in horses. Vet. Immunol Immunopathol. 122, 57-64.
Ward, E. S., 1992. Secretion of T cell receptor fragments from recombinant Escherichia coli cells. J. Mol. Biol. 224: 885-890.
Weber, S., Traunecker, A., Oliveri, F., Gerhard, W., and Karjalainen, K., 1992. Specific low-affinity recognition of major histocompatibility complex plus peptide by soluble T-cell receptor. Nature 356: 793-796.

What is claimed is:

1. A method of making monoclonal antibodies against a protein, comprising generating a fusion protein between said protein and a Th2 cytokine, immunizing a non-human animal with said fusion protein, isolating antibody-producing cells from the immunized animal, and obtaining hybridomas that produce monoclonal antibodies against said protein.

2. The method of claim 1, wherein said Th2 cytokine is selected from the group consisting of IL-4, IL-5, IL-10, IL-13 and IL-31.

3. The method of claim 2, wherein said Th2 cytokine is IL-4.

4. The method of claim 1, wherein said protein is a cell surface receptor protein.

5. The method of claim 1, wherein said protein is selected from the group consisting of T-cell receptor chains, Toll-like receptors, CD23, NK-cell receptors, and tissue factor.

6. The method of claim 1, wherein said protein is a soluble protein.

7. The method of claim 1, wherein said Th2 cytokine is fused to the N-terminus of said protein.

8. The method of claim 1, wherein said fusion protein is recombinantly expressed in a host cell for use in immunization.

9. The method of claim 8, wherein said host cell is selected from bacterial, yeast, insect or mammalian cell.

10. The method of claim 1, wherein said animal is a mammal.

11. The method of claim 10, wherein said mammal is mouse.

12. The method of claim 1, wherein said hybridomas produce IgG.

13. A kit for producing monoclonal antibodies against a protein, comprising an expression vector, wherein said vector comprises from 5′ to 3′, a promoter, a Th2 cytokine coding sequence, a cloning site located at or near the C-terminus of the cytokine coding sequence for insertion of a nucleic acid coding for said protein to form a fusion with said cytokine, and a 3′ transcription termination sequence; a host cell for recombinant expression of said fusion protein, reagents for purification of said fusion protein, immunization instructions, and reagents for screening for specific monoclonal antibodies.

14. An expression vector, comprising a nucleic acid coding for a fusion protein between a protein and a Th2 cytokine.

15. The expression vector of claim 13, wherein said Th2 cytokine is selected from the group consisting of IL-4, IL-5, IL-10, IL-13 and IL-31.

16. The expression vector of claim 13, wherein said Th2 cytokine is fused to the N-terminus of said protein.

17. An isolated fusion protein, comprising a Th2 cytokine fused to a protein of interest.

18. The fusion protein of claim 17, wherein said Th2 cytokine is selected from the group consisting of IL-4, IL-5, IL-10, IL-13 and IL-31.

19. The fusion protein of claim 17, wherein said Th2 cytokine is fused to the N-terminus of said protein.
